(19) United States
(12) Patent Application Publication
(10) Pub. No.: US 2026/0114711 A1
(43) Pub. Date: Apr. 30, 2026

(54) CONTROL SYSTEM FOR A MEDICAL DEVICE

(71) Applicant: The United States Government as represented by the Department of Veterans Affairs, Washington, DC (US)

(72) Inventors: Alexandra Alvarez, Holladay, UT (US); Zack Buono, Frederick, MD (US); Aparna Repaka, Lexington, MA (US)

(21) Appl. No.: 19/368,568

(22) Filed: Oct. 24, 2025

Related U.S. Application Data
(60) Provisional application No. 63/711,774, filed on Oct. 25, 2024.

(51) Int. Cl. A61B 1/00

(52) U.S. Cl. CPC A61B 1/00042; A61B 1/00006; A61B 1/0016

(57) ABSTRACT

A control system for controlling an endoscope control section having at least one dial for operating an endoscope. A first driven gear of the control system includes an inner surface configured to interact with a first dial of the at least one dial of the endoscope control section. A first gear array is configured to drive the first driven gear. A first motor is configured to drive the first gear array. An electronic control module is configured to control the first motor. A user may input command via the electronic control module to operate the endoscope.

### CROSS REFERENCE TO RELATED APPLICATIONS

This application is based upon and claims priority to US Provisional Application No. U.S. 63/711,774, filed Oct. 25, 2024, which application is hereby incorporated by reference herein in its entirety.

### FIELD

This disclosure relates to a control system for a medical device (e.g., a medical scope) having control dials. More specifically, the disclosure relates to an ergonomic control system that can be added on to and can operate a control section of an endoscope.

### BACKGROUND

There are many medical procedures which require the use of a medical scope such as an endoscope. For example, colonoscopy procedures are a common and routine procedure for the screening, diagnosis, and early prevention of a variety of diseases of the lower gastrointestinal tract, such as colon cancer. Approximately 19 million of these procedures are performed in the United States every year. During a typical colonoscopy procedure, a trained clinician, typically a Gastrointestinal (GI) physician, will use an endoscope to visually evaluate the inside of a patient's colon to identify abnormalities, as well as perform a variety of basic capabilities such as biopsies of tissues or simple resections of pre-cancerous or early cancerous growths.

An example endoscope 10 is shown in FIG. 1. As shown in FIG. 1, the endoscope 10 may be composed of a long flexible tube 12, with a camera 14 on the distal end and a control section 16 on the proximal end. The endoscopist may use her right hand to insert the distal end of the scope into a tract of the patient. The endoscopist may use her left hand on the control section 16 of the endoscope 10 to perform various operations such as steering the tip of the endoscope in various directions to guide the scope throughout the tract.

The endoscopist may accomplish this steering by using her left thumb and fingers to rotate two concentric dials, a first dial 18 and a second dial 20 (called angulation knobs), which in turn deflect the endoscope in left/right and up/down directions. This two-dial design for endoscope control was first introduced around 1970 and, despite advances in technology and engineering capabilities, this general design has been altered very little over the past 50+ years. The corresponding mechanism of steering has become standard practice in the field and is taught as an essential base skill for performing the various complex maneuvers for a successful endoscopy. The method of steering the scope, limited by the confines of the scope design, is difficult to perform and takes a significant amount of training to master. In addition, this method of scope control has a significant flaw in that it can result in significant injury to the left thumb of the scope user.

For instance, the left thumb is one of the most common sites of endoscopy related injuries (ERI) in gastroenterologists (GIs) performing colonoscopy. In a survey of 1698 US gastroenterologists, the most common site of ERI was the thumb (overall 60.7% in men and 67.9% in women). 25% reported having a diagnosis of De Quervain's tenosynovitis, also referred to as “colonoscopists' thumb” (compared to 0.5-1.3% prevalence in the general population). This condition is associated with inflammation of the tendons associated with the muscles that assist thumb movements (abductor pollicis longus (APL) and the extensor pollicis brevis). The most common reported mechanism underlying this ERI by gastroenterologists was manipulating the dials in the control section of the colonoscope with the left hand. In an international survey of 368 gastroenterologists, left thumb injury arising from manipulating the dials was one of the most commonly reported ERI (34-35% respondents). Studies evaluating the biomechanics of colonoscopy show that the “Hand Activity levels”, which are a composite of physical exposures on the left hand and thumb significantly exceed safety thresholds and pose a risk for overuse injuries.

These chronic use injuries result in a multitude of negative impacts for the endoscopist end user. Although this significant problem has been identified and highlighted in academic literature, and prior research has been conducted to develop technology approaches to address this issue, there has been little substantial progress in terms of changes to existing technology. The widespread adoption, training, and application of the current scope design and corresponding control techniques throughout the field of endoscopy may have resulted in an “organizational inertia” in which new technologies to address this need cannot easily replace the existing endoscope design. The incentive for hospitals to address this problem by adopting a new technology that fully replaces the current endoscope may not overcome the barriers associated with the adoption of the technology.

While add-on devices, like the device 22 shown in FIGS. 2 and 3, may improve the reach of the first dial 18 farthest away from the thumb, there may be limited or no benefit to procedure time or ease of procedure for physicians having a hand span of less than 19 cm.

There is an opportunity to improve endoscopes and their associated systems and methods of use. Specifically, there is clearly an opportunity to develop devices and/or systems that may reduce or eliminate potential injury to the left thumb of the user of the endoscope while improving or not worsening the functionality of the endoscope.

### SUMMARY

An example control system for controlling an endoscope control section having at least one dial for operating an endoscope is disclosed. The control system comprises a first driven gear comprising an inner surface configured to interact with a first dial of the at least one dial of the endoscope control section. A first gear array is configured to drive the first driven gear. A first motor is configured to drive the first gear array. An electronic control module is configured to control the first motor.

In combination, an example endoscope control section and an example control system for controlling the endoscope control section is disclosed. The endoscope control section comprises a first dial for operating an endoscope. The control system comprises a first driven gear comprising an inner surface configured to interact with the first dial of the at least one dial of the endoscope control section. A first gear array is configured to drive the first driven gear. A first motor is configured to drive the first gear array. An electronic control module is configured to control the first motor.

An example method of using a control system to control an endoscope control section of an endoscope is disclosed. The method comprises inputting commands, via an electronic control module, to control the movement of the endoscope. The electronic control module is configured to control a first motor. The first motor is configured to drive a first gear array. The first gear array is configured to drive a first driven gear. The first driven gear is configured to interact with a first dial of the endoscope control section of the endoscope.

### BRIEF DESCRIPTION OF THE DRAWINGS

FIG. 1 shows an example of a known endoscope;

FIG. 2 shows an example of a known add-on device;

FIG. 3 shows the example add-on device of FIG. 2 and an example known endoscope;

FIG. 4 shows an example control system according to the disclosure;

FIG. 5 shows an example control system according to the disclosure;

FIG. 6 shows an example control system according to the disclosure;

FIG. 7 shows an example control system according to the disclosure;

FIG. 8 shows an example housing of an example control system;

FIG. 9 shows an example housing of an example control system;

FIG. 10 shows an example first housing portion of an example housing;

FIG. 11 shows an example first housing portion of an example housing;

FIG. 12 shows an example electronic control module of an example control system;

FIG. 13 shows an example electronic control module of an example control system;

FIG. 14 shows an example first motor and first gear array of an example control system;

FIG. 15 shows an example second motor and second gear array of an example control system;

FIG. 16 shows an example first driven gear of an example control system;

FIG. 17 shows an example first driven gear of an example control system;

FIG. 18 shows an example second driven gear of an example control system;

FIG. 19 shows an example second driven gear of an example control system;

FIG. 20 shows an example first driven gear and second driven gear of an example control system;

FIG. 21 shows an example first driven gear and second driven gear positioned on an example known endoscope;

FIG. 22 shows an example first driven gear and second driven gear positioned on an example known endoscope;

FIG. 23 shows an example first gear brace of an example control system;

FIG. 24 shows an example first gear brace of an example control system;

FIG. 25 shows an example first gear brace, first gear array, and second gear array of an example control system;

FIG. 26 shows an example first gear brace, first driven gear, first gear array, and second gear array of an example control system;

FIG. 27 shows an example second gear brace of an example control system;

FIG. 28 shows an example second gear brace and second driven gear of an example control system;

FIG. 29 shows an example second gear brace of an example control system;

FIG. 30 shows an example second gear brace of an example control system;

FIG. 31 shows an example first gear brace and second gear brace of an example control system;

FIG. 32 shows an example first gear brace, first driven gear, second gear brace, and second driven gear of an example control system;

FIG. 33 shows an example first driven gear and second driven gear coupled to an example housing of an example control system;

FIG. 34 shows an example first driven gear and second driven gear coupled to an example housing of an example control system;

FIG. 35 shows an example first driven gear and second driven gear coupled to an example housing of an example control system;

FIG. 36 shows an example second housing portion of an example housing;

FIG. 37 shows an example second housing portion of an example housing;

FIG. 38 shows an example cap of an example second housing portion;

FIG. 39 shows an example cap of an example second housing portion;

FIG. 40 shows an example body of an example second housing portion;

FIG. 41 shows an example computing device of an example control system;

FIG. 42 shows a diagram of an example computing device for operating a control system; and

FIG. 43 shows a diagram of an example operating environment.

### DETAILED DESCRIPTION

The present disclosure can be understood more readily by reference to the accompanying detailed description, which includes examples, claims and drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be under-stood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” can optionally include plural referents un-less the context clearly dictates otherwise. For example, use of the term “a gear” can represent disclosure of embodiments in which only a single gear is provided, and unless the context dictates otherwise, can also represent disclosure of embodiments in which a plurality of such gears are provided.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, use of “substantially” (e.g., “substantially parallel”) or “generally” (e.g., “generally planar”) should be understood to include embodiments in which angles are within ten degrees, or within five degrees, or within one degree.

The word “or” as used herein means any one member of a particular list and, in alternative embodiments, unless context dictates otherwise, can include any combination of members of that list.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the device, systems, and associated methods can be implemented and used without employing these specific details. Indeed, the device, systems, and associated methods can be placed into practice by modifying the illustrated device, systems, and associated methods and may be used in conjunction with any other apparatus and techniques conventionally used in the industry.

### Control System

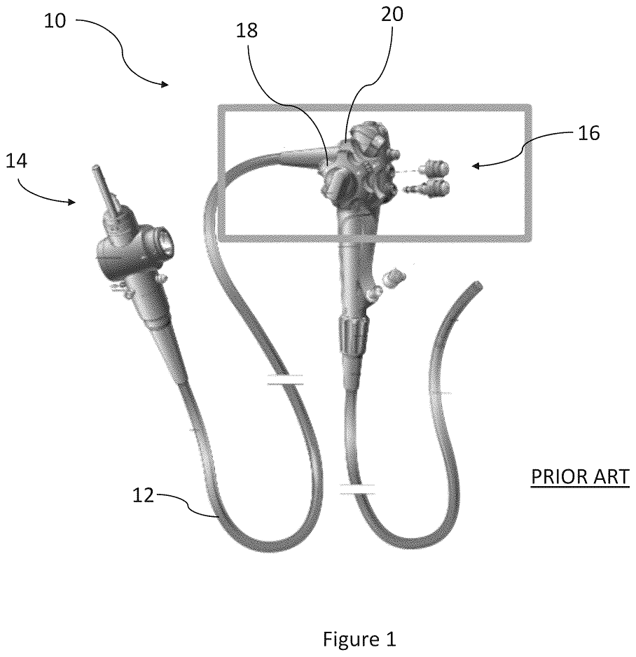

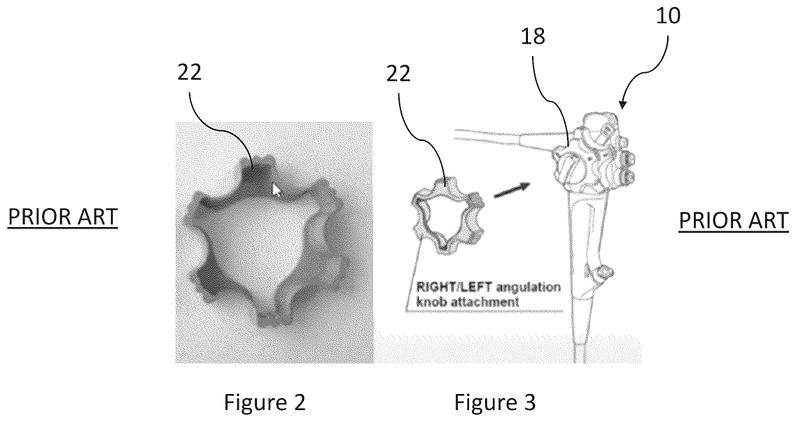

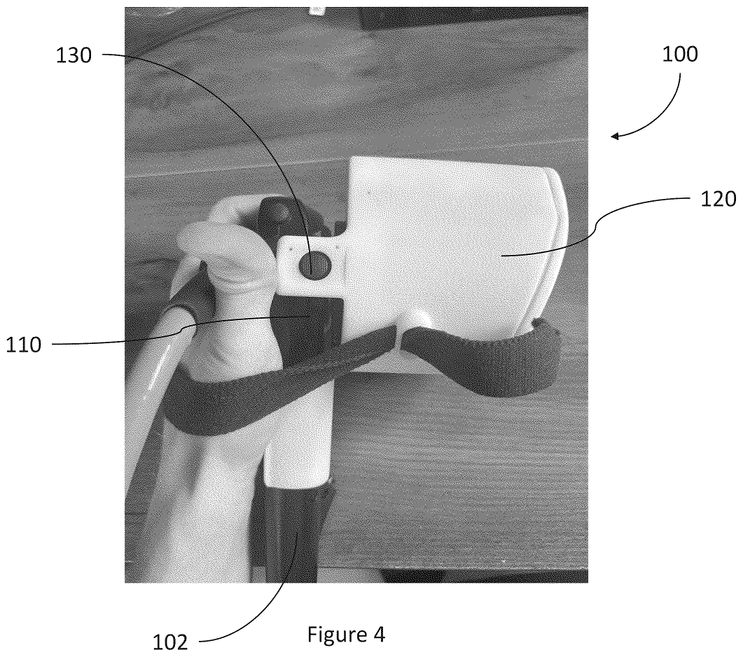

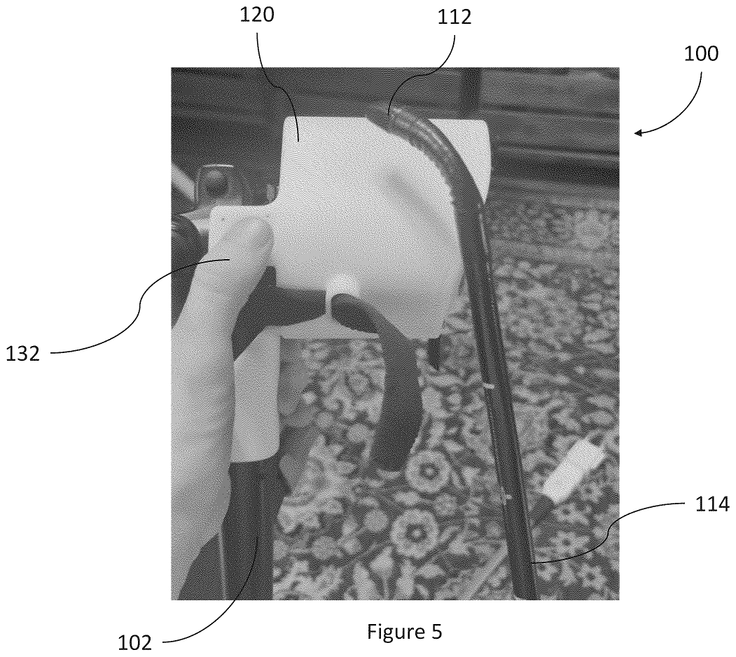

FIGS. 4-7 show an example control system 100 for a medical device (e.g., a medical scope 102) having at least one control dial 104 (shown in FIG. 22). By way of example, the medical scope may be an endoscope such as a colonoscope, cardioscope, duodenum scopes, gastroscope, bronchoscopes, cystoscope, ureteroscopy, arthroscope, etc. In this example, the control system 100 may control an endoscope control section 110 having a first dial 106 and a second dial 108 for operating an endoscope. The medical scope may be an endoscope similar to or identical to the example endoscope 10 shown in FIG. 1. As shown in FIG. 22, the medical scope 102 may comprise a first dial 106 similar to first dial 18 and a second dial 108 similar to second dial 20. The first dial 106 and second dial 108 of the control section 110 may be used to control a distal end portion 112 of the flexible tube 114 of the medical scope 102 (e.g. endoscope) (shown in FIG. 5). For example, the first dial 106 may rotate a first direction and a second, opposite direction about a first dial axis 116 to cause the distal end portion 112 to move in a direction in a first orthogonal plane. For example, the first direction about the first dial axis 116 may correspond to a first direction in the first orthogonal plane and the second direction about the first dial axis 116 may correspond to a second direction in the first orthogonal plane. Optionally, the first orthogonal plane corresponds to a plane orthogonal or substantially orthogonal to an x-axis. The second dial 108 may rotate a first direction and a second, opposite direction about a second dial axis 118 to cause the distal end portion 112 to move in a direction in a second orthogonal plane. For example, the first direction about the second dial axis 118 may correspond to a first direction in the second orthogonal plane and the second direction about the second dial axis 118 may correspond to a second direction in the second orthogonal plane. Optionally, the second orthogonal plane corresponds to a plane orthogonal or substantially orthogonal to a y-axis.

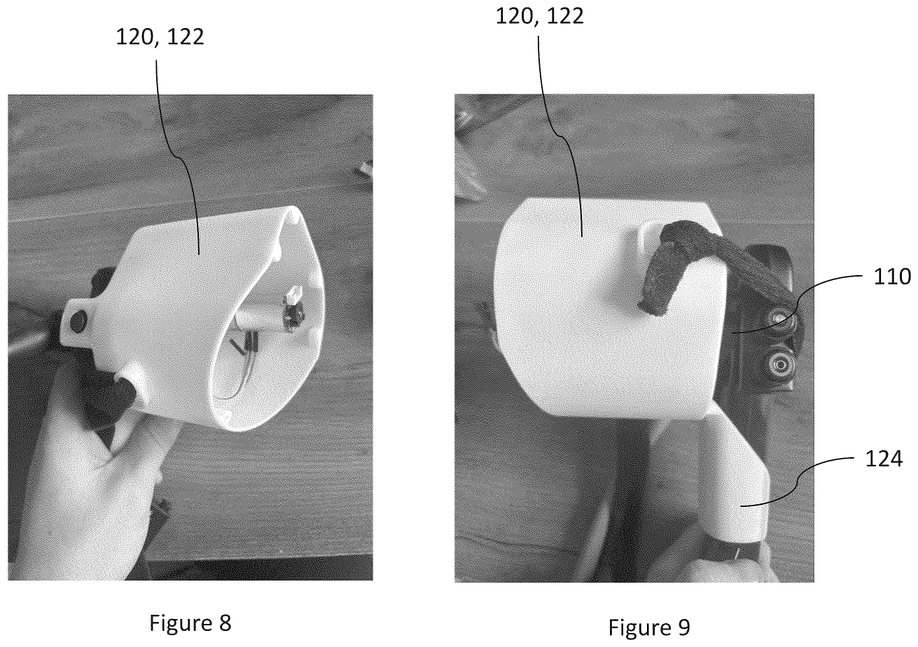

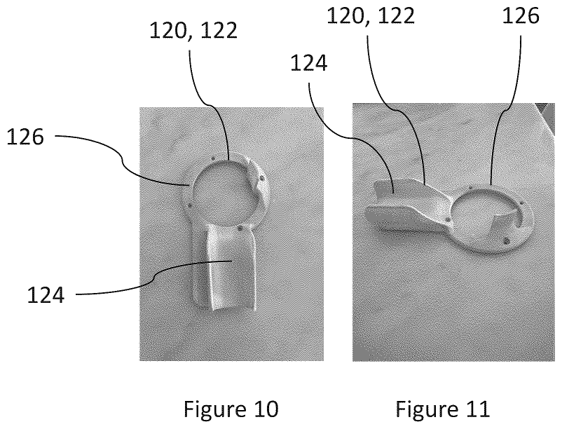

As shown in FIG. 4-11, the system 100 may comprise a housing 120 configured to couple to at least a portion of the scope 102 such that the system 100 may interact with the endoscope control section 110 without interfering with the operation of the scope 102 (e.g. endoscope). The housing 120 may at least partially enclose components of the system 100 to protect the components from the surrounding environment. Optionally, the housing may be waterproof or water resistant to protect the components within the housing 120. As shown in FIGS. 8-11, the housing 120 may comprise a first housing portion 122. Optionally, the first housing portion 122 may be configured to couple to medical scope 102 (e.g. endoscope). Optionally, the first housing portion 122 may comprise an engagement portion 124 configured to engage the medical scope 102 and secure the system 100 to the medical scope 102. The first housing portion 122 may provide a mounting surface 126 to mount components of the system 100.

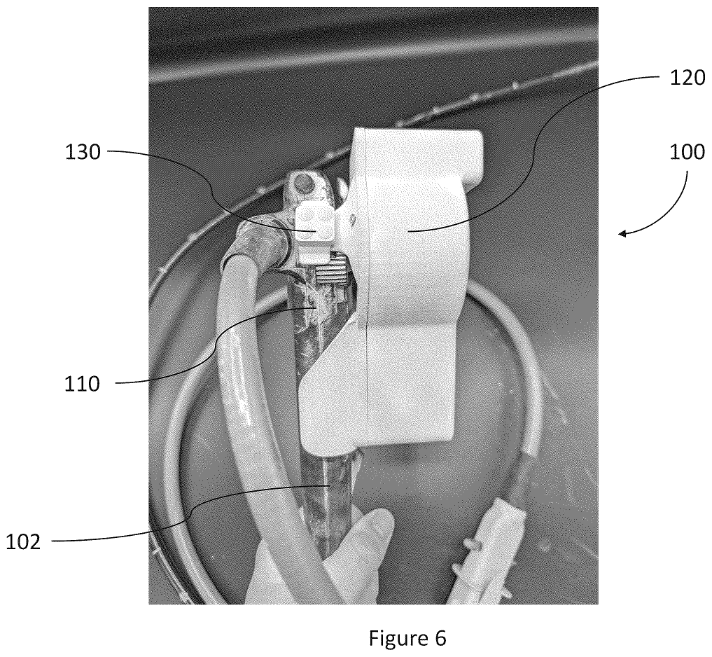

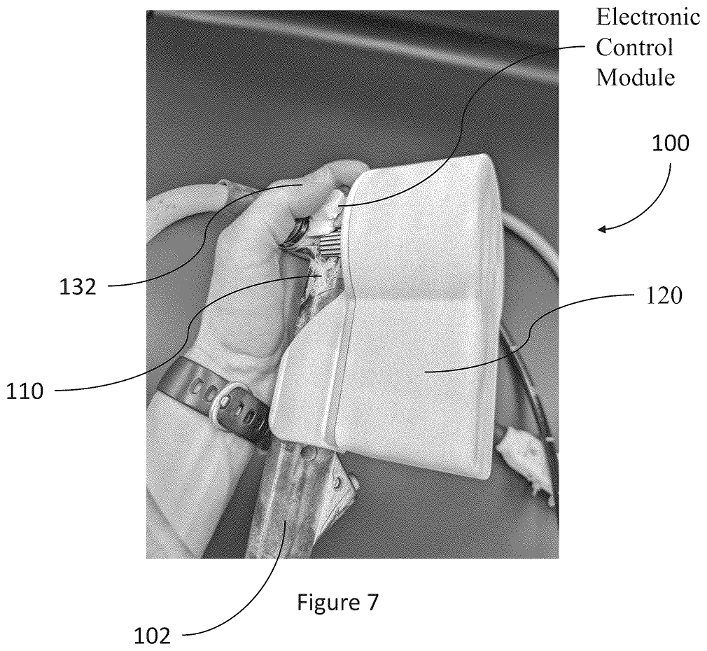

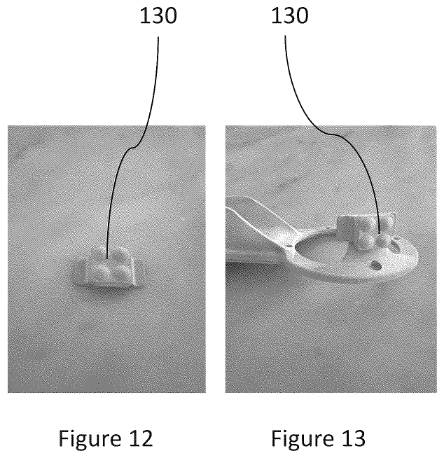

As shown in FIGS. 1, 6, 8, 12, and 13, the system 100 comprises an electronic control module 130. The electronic control module 130 may be at least one of a joystick, a D-pad, or a button array. As shown in FIG. 6, the electronic control module is configured to accept inputs from a user. The inputs may correspond to four directions in two orthogonal planes. The two orthogonal planes may consist of an “x” plane and a “y” plane. The directions and orthogonal planes may correspond to the directions and orthogonal planes in which the distal end portion 112 of the medical scope 102 moves. For example, an input corresponding to a first direction in the “x” plane may correspond to movement of the distal end portion 112 in the first direction in the orthogonal plane in the x-axis. An input corresponding to a second direction in the “x” plane may correspond to movement of the distal end portion 112 in the second direction in the orthogonal plane in the x-axis. An input corresponding to a first direction in the “y” plane may correspond to movement of the distal end portion 112 in the first direction in the orthogonal plane in the y-axis. An input corresponding to a second direction in the “y” plane may correspond to movement of the distal end portion 112 in the second direction in the orthogonal plane in the y-axis. Optionally, the electronic control module 130 may be coupled to the housing 120. Optionally, the electronic control module 130 may be coupled to the first housing portion 122 of the housing 120. Alternatively, the electronic control module may be a separate component from and may not be attached to the housing. The electronic control module 130 may be positioned at an ergonomic position for a thumb 132 of a hand of a user when the hand is holding the housing 120. For example, as shown in FIGS. 5 and 7, the electronic control module 130 may be positioned, optionally, coupled to the housing 120, at a location where the user will not hyperextend the thumb 132 while inputting commands via the electronic control module 130. The position of the electronic control module 130 may reduce the risk of injury of the thumb of the user.

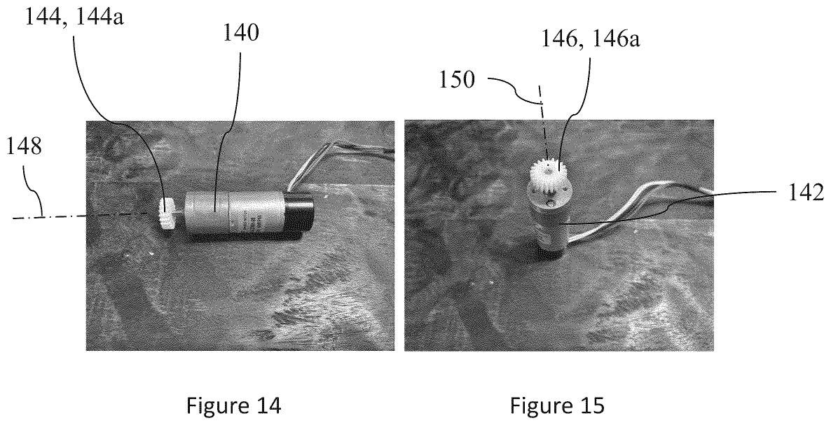

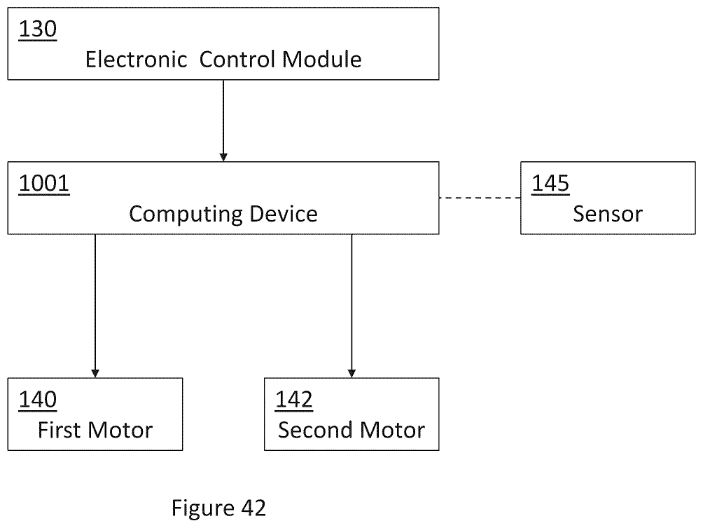

As shown in FIGS. 14 and 15, the system 100 comprises a first motor 140. The system 100 may further comprise a second motor 142. At least one power source may power the first motor 140 and/or the second motor 142. Optionally, the at least one power source may be a battery. Each motor 140, 142 may be a DC, a servo, a stepper, or other standard rotational motor that utilizes an electronic power source to output rotational force. The first motor 140 and/or the second motor 142 may be at least partially positioned within the housing 120. The electronic control module 130 is configured to control the first motor 140 and/or the second motor 142 to control the operation, including movement, of the medical scope 102 (for example, the distal end portion 112 of the endoscope). For example, the electronic control module 130 may cause the first motor 140 to rotate a first direction or a second, opposite direction based on the input received by the electronic control module 130. The electronic control module 130 may cause the second motor 142 to rotate a first direction or a second, opposite direction based on the input received by the electronic control module 130. For example, an input corresponding to the first direction in the “x” plane may cause the first motor 140 to rotate in the first direction. An input corresponding to the second direction in the “x” plane may cause the first motor 140 to rotate in the second direction. An input corresponding to the first direction in the “y” plane may cause the second motor 142 to rotate in the first direction. An input corresponding to the second direction in the “y” plane may cause the second motor 142 to rotate in the second direction. The electronic control module 130 may be connected to the first motor 140 and/or second motor 142 via a wire connection or a wireless connection such as a Bluetooth connection. Optionally, the system 100 may comprise at least one sensor 145 (see FIG. 42). The at least one sensor 145 may be a force or pressure sensor. The force sensor may be integrated into the first motor 140 and/or second motor 142. The at least one sensor 145 may be configured to measure a force applied by the dials to avoid patient perforation or damage from endoscope navigation against bowel walls. This data may be used for feedback and/or control purposes.

The system 100 comprises at least one first gear array 144. The system 100 may comprise at least one second gear array 146. Each gear array comprises at least one gear. Although only one gear is shown in FIGS. 14 and 15, it is contemplated that each gear array 144, 146 may comprise more than one gear. Thus, the first gear array comprises at least a first gear 144a, and the second gear array comprises at least a second gear 146a. Optionally, each gear array axis of the at least one gear array may be parallel or substantially parallel (e.g., within 10 degrees of parallel) to one another or transverse to one another. The first gear array 144 and/or the second gear array 146 may be at least partially positioned within the housing 120. The first motor 140 may be configured to drive the first gear array 144. The first gear array 144 may be configured to transmit the rotational motion of the first motor 140 to a first driven gear 152 as described herein. For example, rotation of the first motor 140 in the first direction may cause the first gear array 144 to rotate in a first direction about a first gear array axis 148. Rotation of the first motor 140 in the second direction may cause the first gear array 144 to rotate in a second direction about the first gear array axis 148. The second motor 142 may be configured to drive the second gear array 146. The second gear array 146 may be configured to transmit the rotational motion of the second motor 142 to a second driven gear 164 as described herein. For example, rotation of the second motor 142 in the first direction may cause the second gear array 146 to rotate in a first direction about a second gear array axis 150. Rotation of the second motor 142 in the second direction may cause the second gear array 146 to rotate in a second direction about the second gear array axis 150.

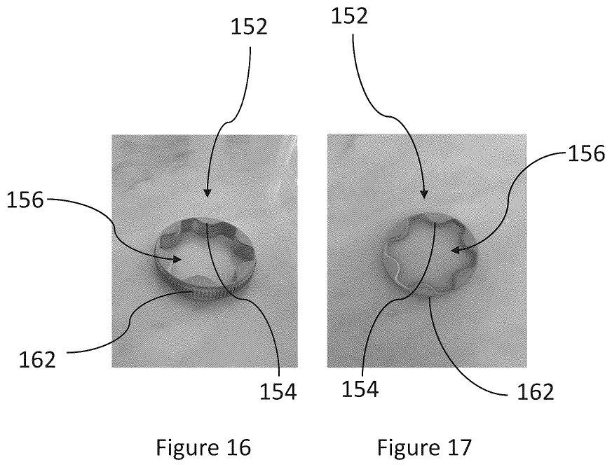

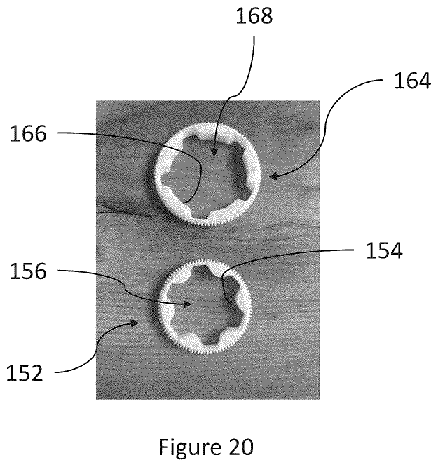

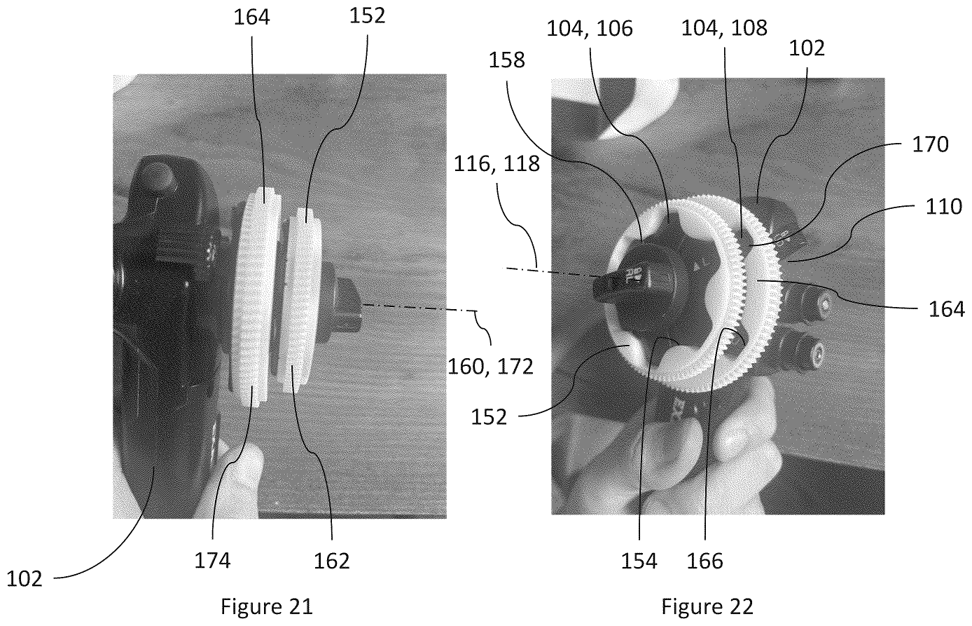

As shown in FIGS. 16, 17, and 20-22, the system 100 comprises a first driven gear 152. The first driven gear 152 may comprise an inner surface 154 configured to interact with the first dial 106 (shown in FIG. 22). The inner surface 154 of the first driven gear 152 may have a first profile defined by a cutout section 156 from the first driven gear 152. The first profile may be complementary to a radially exterior surface 158 of the first dial 106 and the cutout section 156 may correspond to the cross section of the first dial 106 such that the inner surface 154 of the first driven gear 152 is configured to engage the radially exterior surface 158 of the first dial 106 when the system 100 is coupled to the control section 110. Optionally, the inner surface 154 of the first driven gear 152 is configured to continuously engage the radially exterior surface 158 about the entire radially exterior surface 158. The first driven gear 152 is configured to be driven by the first gear array 144. For example, rotation of the first gear array 144 may cause the first driven gear 152 to rotate about a first gear axis 160. Rotation of the first driven gear 152 about the first gear axis 160 may cause the first dial 106 to rotate about the first dial axis 116. Optionally, rotation of the first gear array 144 in the first direction about the first gear array axis 148 may cause the first driven gear 152 to rotate about the first gear axis 160 in a first direction to cause the first dial to rotate about the first dial axis 116 in the first direction. Rotation of the first gear array 144 in the second direction about the first gear array axis 148 may cause the first driven gear 152 to rotate about the first gear axis 160 in a second direction to cause the first dial 106 to rotate about the first dial axis 116 in the second direction. Optionally, the first driven gear 152 comprises a circumferential outer surface 162 comprising teeth configured to interlock with teeth on the outer surface of the first gear array 144.

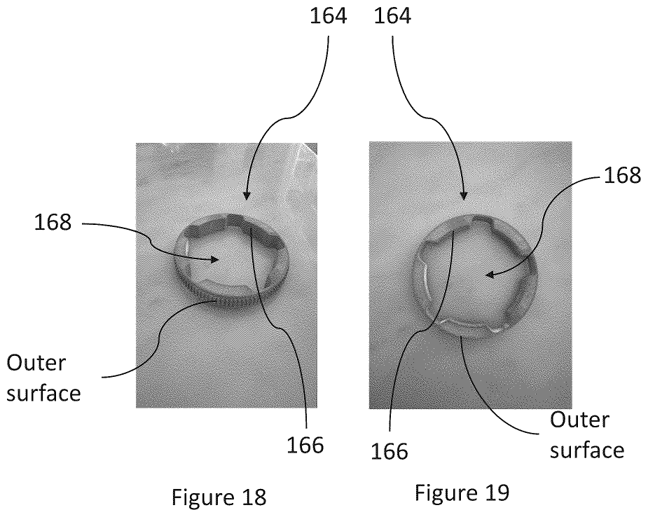

As shown in FIGS. 18-22, the system 100 may comprise a second driven gear 164. The second driven gear 164 may comprise an inner surface 166 configured to interact with the second dial (shown in FIG. 22). The inner surface 166 of the second driven gear 164 may have a second profile defined by a cutout section 168 from the second driven gear 164. The second profile may be complementary to a radially exterior surface 170 of the second dial 108 and the cutout section 168 may correspond to the cross section of the second dial 108 such that the inner surface 166 of the second driven gear 164 is configured to engage the radially exterior surface 170 of the second dial 108 when the system 100 is coupled to the control section 110. Optionally, the inner surface 166 of the second driven gear 164 is configured to continuously engage the radially exterior surface 170 about the entire radially exterior surface 170. The second driven gear 164 is configured to be driven by the second gear array 146. For example, rotation of the second gear array 146 may cause the second driven gear 164 to rotate about a second gear axis 172. Rotation of the second driven gear 164 about the second gear axis 172 may cause the second dial 108 to rotate about the second dial axis 118. Optionally, rotation of the second gear array 146 in the first direction about the first gear array axis 150 may cause the second driven gear 164 to rotate about the second gear axis 172 in a first direction to cause the second dial 108 to rotate about the second dial axis 118 in the first direction. Rotation of the second gear array 146 in the second direction about the second gear array axis 150 may cause the second driven gear 164 to rotate about the second gear axis 172 in a second direction to cause the second dial 108 to rotate about the second dial axis 118 in the second direction. Optionally, the second driven gear 164 comprises a circumferential outer surface 174 comprising teeth configured to interlock with teeth on the outer surface of the second gear array 146. The first driven gear 152 and/or the second driven gear 164 may be positioned within the housing 120.

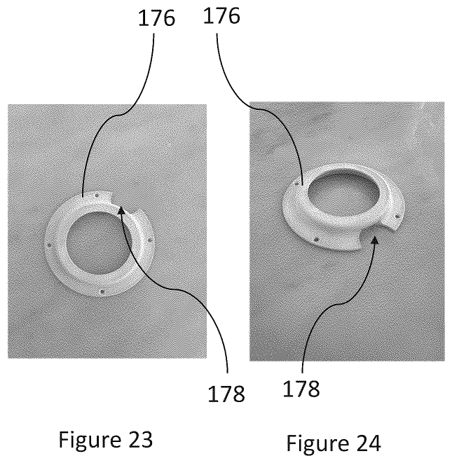

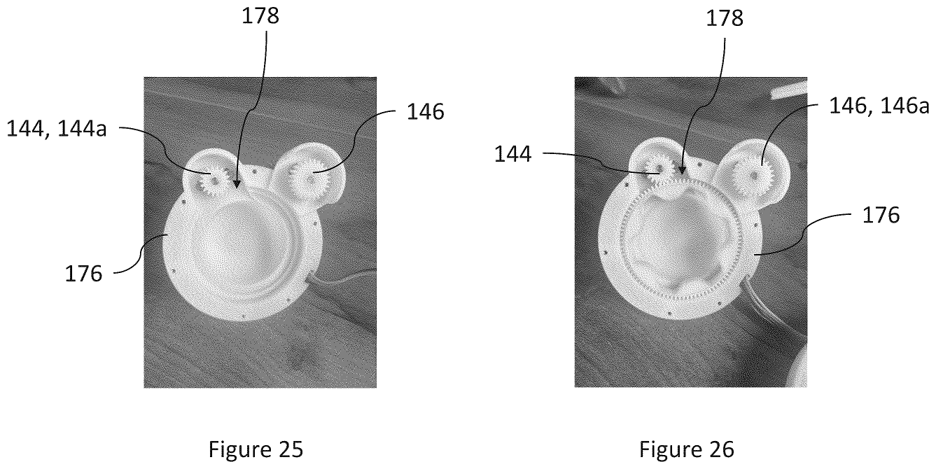

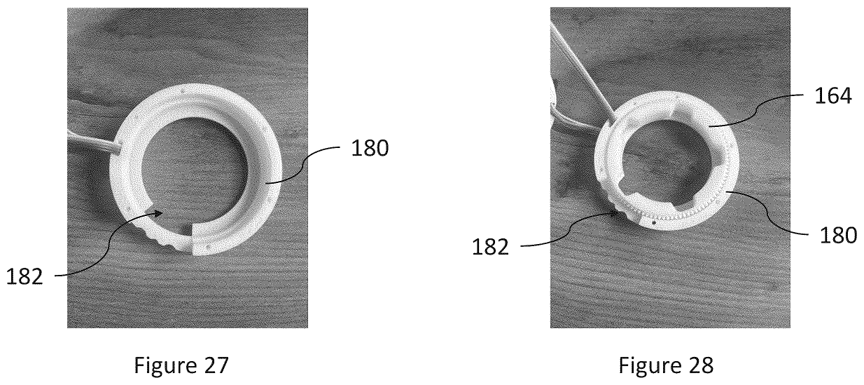

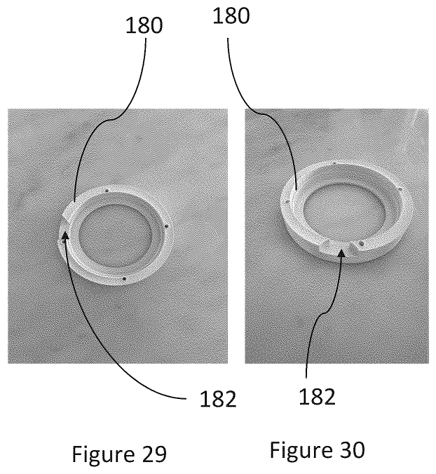

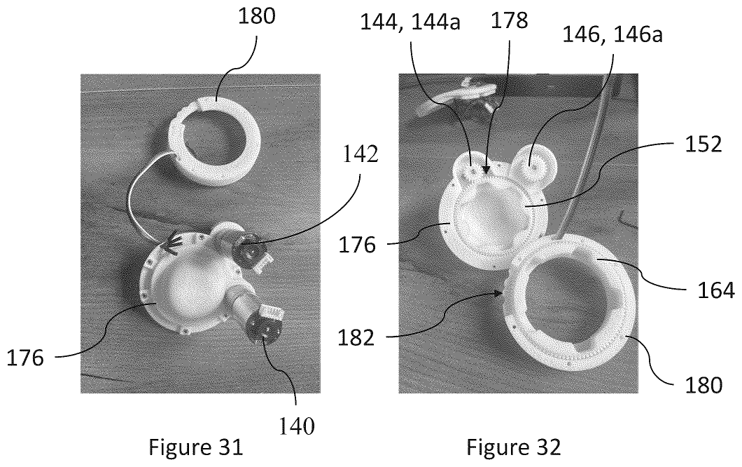

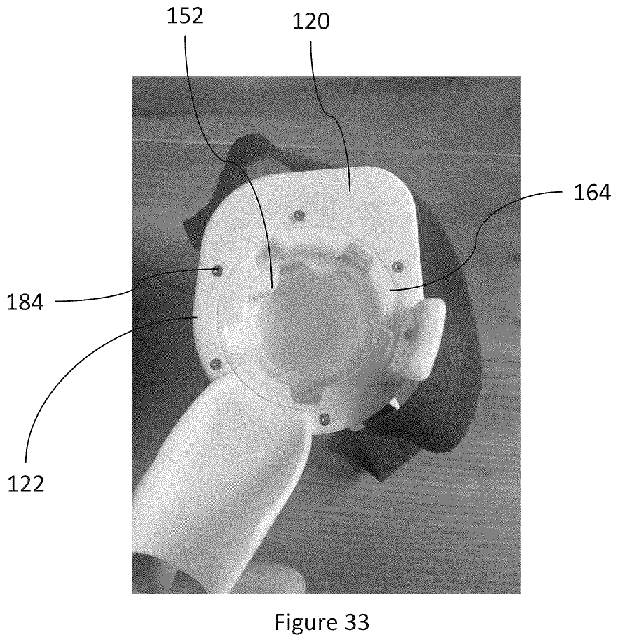

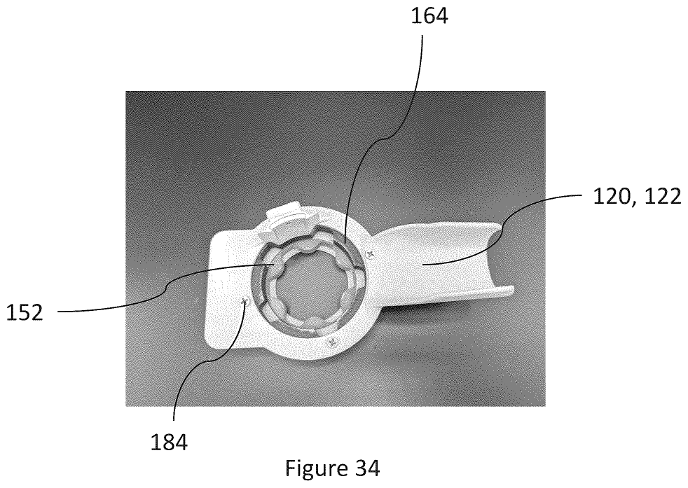

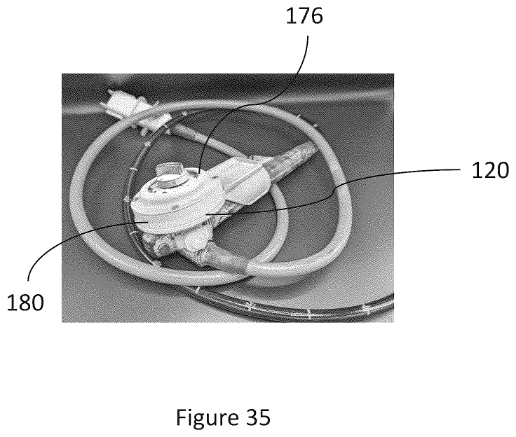

As shown in FIGS. 23-26, 31, and 32, the system 100 may comprise a first gear brace 176. The first gear brace 176 may be configured to couple the first driven gear 152 to the housing 120 to stabilize radial alignment of the first driven gear 152 while allowing the first driven gear 152 to freely rotate. The first gear brace 176 may comprise a first cutout 178 or opening to allow the first gear array 144 to interact with the first driven gear 152 (as shown in FIG. 32). As shown in FIGS. 27-32, the system 100 may comprise a second gear brace 180 configured to couple the second gear 164 to the housing 120 to stabilize radial alignment of the second driven gear 164 while allowing the second driven gear 164 to freely rotate. The second gear brace 180 may comprise a second cutout 182 or opening to allow the second gear array 146 to interact with the second driven gear 164. As shown in FIGS. 33 and 34, optionally, the first gear brace 176 and/or second gear brace 180 may be coupled to the housing 120 (optionally, the first housing portion 122 of the housing) via at least one fastener 184. As shown in FIG. 35, the first gear brace 176 may be coupled to the housing 120 via the second gear brace 180. For example, the first gear brace 176 may coupled to the second gear brace 180.

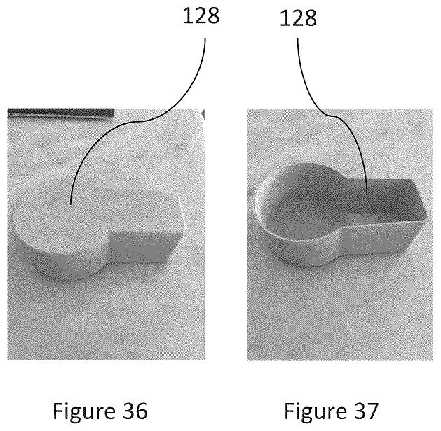

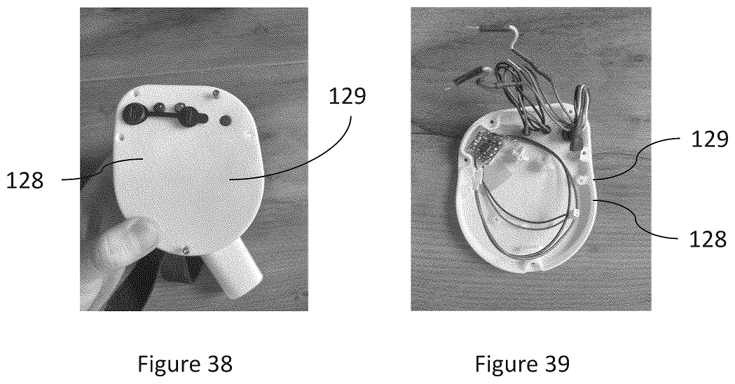

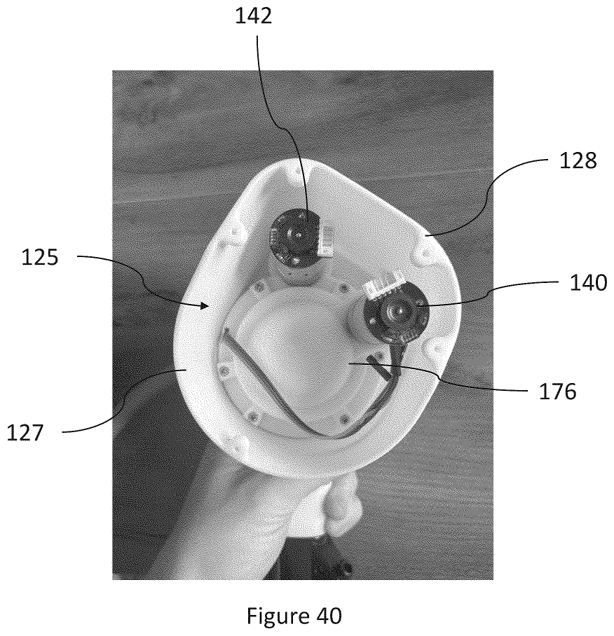

As shown in FIGS. 36-40, the housing 120 may comprise a second housing portion 128 configured to enclose at least some of the components of the system 100. Optionally, as shown in FIG. 40, the second housing portion 128 may comprise a body 127 defining a compartment 125 for at least some of the components of the system 100. As shown in FIGS. 38 and 39, the second housing portion 128 may comprise a cap 129 configured to be coupled to the body 127 to enclose the components positioned within the compartment 125 of the body 127. Optionally, the second housing portion 128 may couple to the first housing portion 122. Optionally, the second housing portion 128 may couple to the medical scope 102 (e.g. endoscope).

### Computing Device

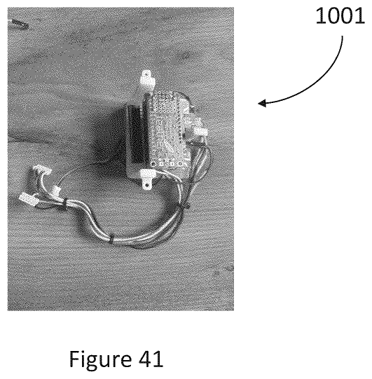

As shown in FIGS. 41 and 42, the system 100 may comprise at least one computing device 1001 operably coupled to the electronic control module 130 and the first motor 140. The computing device 1001 may be operably coupled to the second motor 142. The at least one computing device 1001 may be at least one of an Arduino Nano, micro-computer, custom Printed Circuit Board, and similar electronic control board. One or more computing device 1001 can control a plurality of operations, including: receiving a signal from the electronic control module 1010; controlling the rotational output of the first motor 140; and controlling the rotation output of the second motor 142. The computing device 1001 may be configured to receive data from the at least one sensor 145 (e.g. force or pressure sensor). The computing device 1001 may be configured to compare the measured force via the at least one sensor to a threshold force. The at least one computing device 1001 may be configured to prevent the first motor and/or second motor from rotating or cause the first motor and/or second motor to rotate to move the end portion 112 of the endoscope patient tissue (e.g. bowel walls) if the measured force exceeds the threshold force. In some optional aspects, a single computing device 1001 controls a plurality of such operations. In some aspects, the system can comprise a plurality of computing devices 1001 that operate in coordination. For example, a first computing device 1001 may receive the signal corresponding to the user input via the electronic control module 130. Still another computing device 1001 may cause the first motor 140 or the second motor 142 to rotate a rotational direction corresponding to the received signal. Each of the computing devices can optionally be embodied in accordance with the computing device 1001 as further disclosed herein.

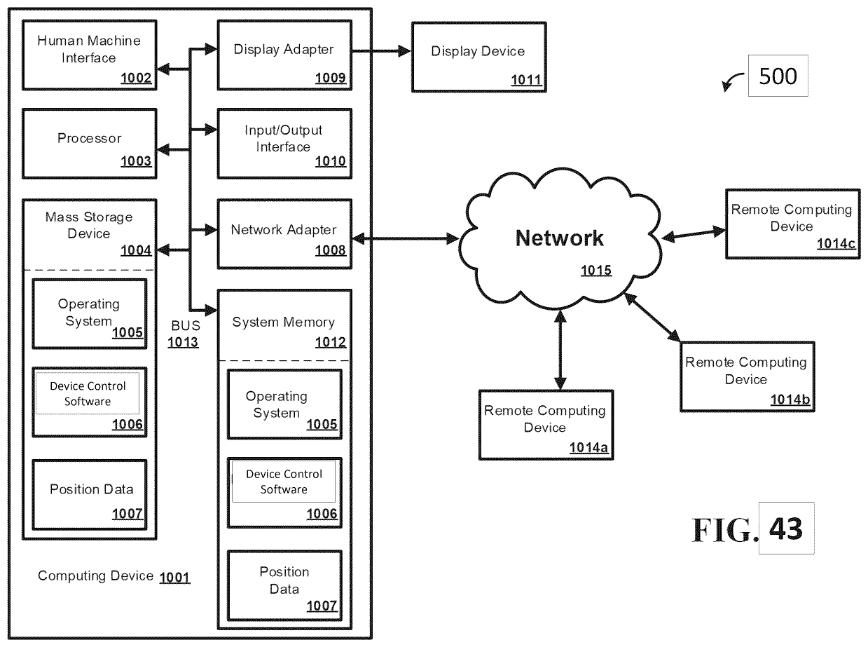

FIG. 43 shows an example operating environment 500 including an exemplary configuration of a computing device 1001 for use with the system disclosed herein. The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing. The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as position data 1007 and/or program modules such as operating system 1005, and device control software 1006 for controlling the motors and/or components of the system.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like. Any number of program modules may be stored on the mass storage device 1004. An operating system 1005 and software 1006 may be stored on the mass storage device 1004.

A user may enter commands and information into the computing device 1001 using an input device such as the electronic control module 130. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. The remote computing devices 1014a,b,c, can perform respective operations of the system. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### Operation of the System

The computing device 1001 may be configured to receive a signal corresponding to the user input and control the first motor and the second motor based on the signal. For example, the computing device 1001 may be configured to cause the first motor 140 to rotate in a first rotational direction or a second rotational direction opposite the first rotational direction about a first motor axis based on the signal. The computing device 1001 may be configured to cause the second motor 142 to rotate in a first rotational direction or a second rotational direction opposite the first rotational direction about a second motor axis based on the signal. The first gear array 144 may transmit rotation of the first motor 140 in the first rotational direction to drive the first driven gear 152 in a first rotational direction about a first gear axis 160 and may transmit rotation of the first motor 140 in the second rotational direction to drive the first driven gear 152 in a second rotational direction about the first gear axis 160. The second gear array 146 may transmit rotation of the second motor 142 in the first rotational direction to drive the second driven gear 164 in a first rotational direction about a second gear axis 172 and may transmit rotation of the second motor 142 in the second rotational direction to drive the second driven gear 164 in a second rotational direction about the second gear axis 172. Rotation of the first driven gear 152 in the first rotational direction about the first gear axis 160 may cause the first dial 106 to rotate in a first rotational direction about a first dial axis 116. The first dial axis 116 may be arranged coaxially with the first gear axis 160. Rotation of the first driven gear 152 in the second rotational direction about the first gear axis 160 may cause the first dial 106 to rotate in a second rotational direction about the first dial axis 116. Rotation of the second driven gear 164 in the first rotational direction about the second gear axis 172 may cause the second dial 108 to rotate in a first rotational direction about a second dial axis 118. The second dial axis 118 may be arrange coaxially with the second gear axis 172. Rotation of the second driven gear 164 in the second rotational direction about the second gear axis 172 may cause the second dial 108 to rotate in a second rotational direction about the second dial axis 118. The first rotational direction of the first dial 106 may cause the endoscope to move in a first direction in a first orthogonal plane and the second rotational direction of the first dial 106 may cause the endoscope to move in a second direction in the first orthogonal plane. The first rotational direction of the second dial 108 may cause the endoscope to move in a first direction in a second orthogonal plane and the second rotational direction of the second dial 108 may cause the endoscope to move in a second direction in the second orthogonal plane.

Control systems according to the disclosure are expected to reduce the biomechanical workload of the thumb of a user performing a procedure utilizing an endoscope such as a colonoscope. It is expected that control systems according to the disclosure may reduce the risk of thumb injury of endoscopists while the majority of the endoscopy process remains unchanged and the endoscope's functionality may be unaffected. Further, the control system may be easy to learn and use.

### EXEMPLARY ASPECTS

In view of the described device, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used herein.

Aspect 1: A control system for controlling an endoscope control section having at least one dial for operating an endoscope, the control system comprising:
a first driven gear comprising an inner surface configured to interact with a first dial of the at least one dial of the endoscope control section;
a first gear array configured to drive the first driven gear;
a first motor configured to drive the first gear array; and
an electronic control module configured to control the first motor.

Aspect 2: The control system according to aspect 1 further comprising:
a second gear comprising an inner surface configured to interact with a second dial of the at least one dial of the endoscope control section;
a second gear array configured to drive the second gear; and
a second motor configured to drive the second gear array, wherein
the electronic control module is configured to control the second motor.

Aspect 3: The control system according to aspect 1 or aspect 2, wherein the electronic control module is configured to accept inputs from a user corresponding to four directions in two orthogonal planes.

Aspect 4: The control system according to aspects 3 further comprising a computing device operably coupled to the electronic control module, the first motor, and the second motor.

Aspect 5: The control system according to aspect 4, wherein the computing device is configured to receive a signal corresponding to the user input and control the first motor and the second motor based on the signal.

Aspect 6: The control system according to aspect 5, wherein the computing device is configured to cause the first motor to rotate in a first rotational direction or a second rotational direction opposite the first rotational direction about a first motor axis based on the signal, and the computing device is configured to cause the second motor to rotate in a first rotational direction or a second rotational direction opposite the first rotational direction about a second motor axis based on the signal.

Aspect 7: The control system according to aspect 6, wherein the first gear array is configured to transmit rotation of the first motor in the first rotational direction to drive the first driven gear in a first rotational direction about a first gear axis and is configured to transmit rotation of the first motor in the second rotational direction to drive the first driven gear in a second rotational direction about the first gear axis, and
wherein the second gear array is configured to transmit rotation of the second motor in the first rotational direction to drive the second gear in a first rotational direction about a second gear axis and is configured to transmit rotation of the second motor in the second rotational direction to drive the second gear in a second rotational direction about the second gear axis.

Aspect 8: The control system according to aspect 7, wherein rotation of the first driven gear in the first rotational direction about the first gear axis causes the first dial to rotate in a first rotational direction about a first dial axis and rotation of the first driven gear in the second rotational direction about the first gear axis causes the first dial to rotate in a second rotational direction about the first dial axis; and
wherein rotation of the second gear in the first rotational direction about the second gear axis causes the second dial to rotate in a first rotational direction about a second dial axis, and rotation of the second gear in the second rotational direction about the second gear axis causes the second dial to rotate in a second rotational direction about the second dial axis.

Aspect 9: The control system according to aspect 8, wherein the first rotational direction of the first dial causes the endoscope to move in a first direction in a first orthogonal plane of the two orthogonal planes and the second rotational direction of the first dial causes the endoscope to move in a second direction in the first orthogonal plane; and
wherein the first rotational direction of the second dial causes the endoscope to move in a first direction in a second orthogonal plane of the two orthogonal planes and the second rotational direction of the second dial causes the endoscope to move in a second direction in the second orthogonal plane.

Aspect 10: The control system according to any of the preceding aspects, wherein the first driven gear comprises an inner surface having a first profile defined by a cutout section from the first driven gear, wherein the first profile is complementary to a radially exterior surface of the first dial and the cutout section corresponds a cross section of the first dial.

Aspect 11: The control system according to any of aspects 2-10, wherein the second gear comprises an inner surface having a second profile defined by a cutout section from the second gear, wherein the second profile is complementary to a radially exterior surface of the second dial and the cutout section corresponds to a cross section of the second dial.

Aspect 12: The control system according to any of aspects 2-11 further comprising a housing configured to attach to at least a portion of the endoscope, wherein the first driven gear, the second driven gear, the first gear array, and the second gear array are positionable within the housing.

Aspect 13: The control system according to aspect 12, wherein the first motor and the second motor are at least partially positionable within the housing.

Aspect 14: The control system according to aspect 12 or aspect 13, wherein the electronic control module is coupled to the housing.

Aspect 15: The control system according to any of aspects 12-14 further comprising:
a first gear brace configured to couple the first driven gear to the housing to stabilize radial alignment of the first driven gear while allowing the first driven gear to freely rotate, and
a second gear brace configured to couple the second gear to the housing to stabilize radial alignment of the second gear while allowing the second gear to freely rotate.

Aspect 16: The control system according to aspect 15, wherein the first gear brace comprises a first cutout to allow the first gear array to interact with the first driven gear, and
the second gear brace comprises a second cutout to allow the second gear array to interact with the second gear.

Aspect 17: In combination, an endoscope control section and a control system for controlling the endoscope control section, the endoscope control section comprising a first dial for operating an endoscope,
the control system comprising:
a first driven gear comprising an inner surface configured to interact with the first dial of the at least one dial of the endoscope control section;
a first gear array configured to drive the first driven gear;
a first motor configured to drive the first gear array; and
an electronic control module configured to control the first motor.

Aspect 18: The combination according to aspect 17, wherein the endoscope control section further comprises a second dial for operating the endoscope, and wherein the control system further comprising:
a second gear comprising an inner surface configured to interact with the second dial of the at least one dial of the endoscope control section;
a second gear array configured to drive the second gear; and
a second motor configured to drive the second gear array, wherein
the electronic control module is configured to control the second motor.

Aspect 19: The combination according to aspect 18, wherein the electronic control module is configured to accept inputs from a user corresponding to four directions in two orthogonal planes, wherein the control system further comprises a computing device operably coupled to the electronic control module, the first motor, and the second motor, wherein the computing device is configured to receive a signal corresponding to the user input and control the first motor and the second motor based on the signal to drive the first driven gear and the second driven gear to operate the first dial and the second dial.

Aspect 20: A method of using a control system to control an endoscope control section of an endoscope, the method comprising:
inputting commands, via an electronic control module, to control the movement of the endoscope, wherein the electronic control module controls a first motor, wherein the first motor drives a first gear array, wherein the first gear array drives a first driven gear, wherein the first driven gear interacts with a first dial of the endoscope control section of the endoscope.

What is claimed is:

1. A control system for controlling an endoscope control section having at least one dial for operating an endoscope, the control system comprising:
a first driven gear comprising an inner surface configured to interact with a first dial of the at least one dial of the endoscope control section;
a first gear array configured to drive the first driven gear;
a first motor configured to drive the first gear array; and
an electronic control module configured to control the first motor.

2. The control system according to claim 1, further comprising:
a second driven gear comprising an inner surface configured to interact with a second dial of the at least one dial of the endoscope control section;
a second gear array configured to drive the second driven gear; and
a second motor configured to drive the second gear array, wherein
the electronic control module is configured to control the second motor.

3. The control system according to claim 2, wherein the electronic control module is configured to accept inputs from a user corresponding to four directions in two orthogonal planes.

4. The control system according to claim 3 further comprising a computing device operably coupled to the electronic control module, the first motor, and the second motor.

5. The control system according to claim 4, wherein the computing device is configured to receive a signal corresponding to the user input and control the first motor and the second motor based on the signal.

6. The control system according to claim 5, wherein the computing device is configured to cause the first motor to rotate in a first rotational direction or a second rotational direction opposite the first rotational direction about a first motor axis based on the signal, and the computing device is configured to cause the second motor to rotate in a first rotational direction or a second rotational direction opposite the first rotational direction about a second motor axis based on the signal.

7. The control system according to claim 6, wherein the first gear array is configured to transmit rotation of the first motor in the first rotational direction to drive the first driven gear in a first rotational direction about a first gear axis and is configured to transmit rotation of the first motor in the second rotational direction to drive the first driven gear in a second rotational direction about the first gear axis, and
wherein the second gear array is configured to transmit rotation of the second motor in the first rotational direction to drive the second driven gear in a first rotational direction about a second gear axis and is configured to transmit rotation of the second motor in the second rotational direction to drive the second driven gear in a second rotational direction about the second gear axis.

8. The control system according to claim 7, wherein rotation of the first driven gear in the first rotational direction about the first gear axis causes the first dial to rotate in a first rotational direction about a first dial axis and rotation of the first driven gear in the second rotational direction about the first gear axis causes the first dial to rotate in a second rotational direction about the first dial axis; and
wherein rotation of the second driven gear in the first rotational direction about the second gear axis causes the second dial to rotate in a first rotational direction about a second dial axis, and rotation of the second driven gear in the second rotational direction about the second gear axis causes the second dial to rotate in a second rotational direction about the second dial axis.

9. The control system according to claim 8, wherein the first rotational direction of the first dial causes the endoscope to move in a first direction in a first orthogonal plane of the two orthogonal planes and the second rotational direction of the first dial causes the endoscope to move in a second direction in the first orthogonal plane; and
wherein the first rotational direction of the second dial causes the endoscope to move in a first direction in a second orthogonal plane of the two orthogonal planes and the second rotational direction of the second dial causes the endoscope to move in a second direction in the second orthogonal plane.

10. The control system according to claim 1, wherein the first driven gear comprises an inner surface having a first profile defined by a cutout section from the first driven gear, wherein the first profile is complementary to a radially exterior surface of the first dial and the cutout section corresponds a cross section of the first dial.

11. The control system according to claim 2, wherein the second driven gear comprises an inner surface having a second profile defined by a cutout section from the second driven gear, wherein the second profile is complementary to a radially exterior surface of the second dial and the cutout section corresponds to a cross section of the second dial.

12. The control system according to claim 2, further comprising a housing configured to attach to at least a portion of the endoscope, wherein the first driven gear, the second driven gear, the first gear array, and the second gear array are positionable within the housing.

13. The control system according to claim 12, wherein the first motor and the second motor are at least partially positionable within the housing.

14. The control system according to claim 12, wherein the electronic control module is coupled to the housing.

15. The control system according to claim 12, further comprising:
a first gear brace configured to couple the first driven gear to the housing to stabilize radial alignment of the first driven gear while allowing the first driven gear to freely rotate, and
a second gear brace configured to couple the second driven gear to the housing to stabilize radial alignment of the second driven gear while allowing the second driven gear to freely rotate.

16. The control system according to claim 15, wherein the first gear brace comprises a first cutout to allow the first gear array to interact with the first driven gear, and
the second gear brace comprises a second cutout to allow the second gear array to interact with the second driven gear.

17. In combination, an endoscope control section and a control system for controlling the endoscope control section, the endoscope control section comprising a first dial for operating an endoscope,
the control system comprising:
a first driven gear comprising an inner surface configured to interact with the first dial of the at least one dial of the endoscope control section;
a first gear array configured to drive the first driven gear;
a first motor configured to drive the first gear array; and
an electronic control module configured to control the first motor.

18. The combination according to claim 17, wherein the endoscope control section further comprises a second dial for operating the endoscope, and wherein the control system further comprising:
a second driven gear comprising an inner surface configured to interact with the second dial of the at least one dial of the endoscope control section;
a second gear array configured to drive the second driven gear; and
a second motor configured to drive the second gear array, wherein
the electronic control module is configured to control the second motor.

19. The combination according to claim 18, wherein the electronic control module is configured to accept inputs from a user corresponding to four directions in two orthogonal planes, wherein the control system further comprises a computing device operably coupled to the electronic control module, the first motor, and the second motor, wherein the computing device is configured to receive a signal corresponding to the user input and control the first motor and the second motor based on the signal to drive the first driven gear and the second driven gear to operate the first dial and the second dial.

20. A method of using a control system to control an endoscope control section of an endoscope, the method comprising:
inputting commands, via an electronic control module, to control the movement of the endoscope, wherein the electronic control module controls a first motor, wherein the first motor drives a first gear array, wherein the first gear array drives a first driven gear, wherein the first driven gear interacts with a first dial of the endoscope control section of the endoscope.
